(19) United States
(12) Patent Application Publication
(10) Pub. No.: US 2010/0222652 A1
(43) Pub. Date: Sep. 2, 2010

(54) DIAGNOSTIC SENSOR UNIT

(76) Inventors: Ok Kyung Cho, Schwerte (DE); Yoon Ok Kim, Schwerte (DE)

Correspondence Address: COLLARD & ROE, P.C., 1077 NORTHERN BOULEVARD, ROSLYN, NY, 11576, US

(21) Appl. No.: 12/733,549

(22) PCT Filed: Sep. 8, 2008
(86) PCT No.: PCT/EP2008/007330

§ 371 Date: Apr. 30, 2010

(30) Foreign Application Priority Data
Sep. 7, 2007 (DE) 10 2007 042 551.3

(51) Int. Cl. A61B 5/00

(52) U.S. Cl. 600/301

(57) ABSTRACT

The invention relates to a diagnostic sensor unit for the non-invasive detection of at least one physiological parameter of body tissue near the surface of the skin. The diagnostic sensor unit comprises an optical measurement unit (100) having at least one radiation source (4) for irradiating the tissue to be examined and at least one radiation sensor (5) for detecting the radiation scattered and/or transmitted by the tissue, and an EKG unit (132) for detecting an EKG signal via two or more EKG electrodes (7), wherein at least one radiation source (4) and at least one radiation sensor (5) of the optical measurement unit are disposed in a common sensor housing (400) and wherein at least one EKG electrode (7) of the EKG unit (132) is disposed on the housing surface of the sensor housing (400), specifically such that the EKG electrode (7) comes into contact with the surface of the skin in the area of body tissue detected by the optical measurement unit (100).

The invention relates to a diagnostic sensor unit for non-invasive detection of at least one physiological parameter of body tissue close to the skin surface, having an optical measurement unit that comprises at least one radiation source for irradiating the body tissue to be examined, and at least one radiation sensor for detecting the radiation scattered and/or transmitted by the body tissue, whereby the at least one radiation source and the at least one radiation sensor are disposed in a common sensor housing.

Providing oxygen to body tissue is known to belong to the most important vital functions of human beings. For this reason, oximetry diagnosis modalities are of great importance in medicine nowadays. So-called pulse oximeters are routinely used. The diagnostic sensor unit of such pulse oximeters typically comprises an optical measurement unit with two light sources, which radiate red or infrared light of different wavelengths into the body tissue. The light is scattered in the body tissue and partly absorbed. The scattered light is finally detected by means of a light sensor in the form of a suitable photo cell (photo diode). Typically, commercial pulse oximeters use light in the wavelength range of 660 nm, for one thing. In this range, the light absorption of oxyhemoglobin and deoxyhemoglobin is very different. Accordingly, the intensity of the scattered light detected by means of the photo sensor varies as a function of how strongly the body tissue being examined is perfused by oxygen-rich or oxygen-poor blood. For another thing, light in the wavelength range of 810 nm is usually used. This light wavelength lies in the so-called near infrared spectral range. The light absorption of oxyhemoglobin and deoxyhemoglobin is essentially the same in this spectral range. The known pulse oximeters are furthermore able to generate a plethysmographic signal, i.e. a volume pulse signal, which reproduces the changing amount of blood during a heartbeat, in the microvascular system covered by the pulse oximeter (so-called photoplethysmography). When using different light wavelengths in the spectral ranges indicated above, it is possible to draw conclusions concerning the oxygen content of the blood (oxygen saturation) from the different light absorption. The usual pulse oximeters are used either on the fingertip of a patient or also on the earlobe. Then, the volume pulse signal is generated from the blood perfusion of the microvascular system in these regions of the body tissue.

An EKG (electrocardiogram) is probably the examination modality most frequently used for diagnosis of cardiovascular illnesses. By means of the diagnostic sensor unit of an EKG device, electrical signals are derived from the body of the patient to be examined, using two or more EKG electrodes. The EKG obtained in this manner reproduces the bioelectrical voltages that occur in the heart during excitation propagation and regression. The EKG contains numerous parameters that can be evaluated diagnostically. At the time of contraction of the heart muscle during a heartbeat, the EKG shows a clear peak, which is also called the R peak. Furthermore, the EKG contains the so-called P wave that precedes the R peak. The R peak is followed, in turn, by the so-called T wave. The minima in the EKG directly before and directly after the R peak are referred to as Q and S, respectively. Parameters that are of interest for cardiovascular diagnosis are the duration of the P wave as well as the amplitude of the P wave, the duration of the PQ interval, the duration of the QRS complex, the duration of the QT interval, as well as the amplitude of the T wave. Conclusions concerning the state of health of the cardiovascular system can be drawn both from the absolute values of the aforementioned parameters and from the ratios of the parameters.

It has recently become known that the combined use of different diagnosis modalities, for example pulse oximetry with EKG measurement, is particularly advantageous, in order to obtain information about the state of health of the patient with regard to possible illnesses of the cardiovascular system and possible metabolic illnesses in fast and reliable manner, for example within the framework of health screenings.

Against this background, the present invention is based on the task of making available a diagnostic sensor unit for non-invasive determination of physiological parameters, whose functionality is expanded as compared with the state of the art. In particular, a sensor unit is supposed to be created that can be produced in cost-advantageous manner, on the one hand, and on the other hand can be conveniently and easily used by the user to allow reliable and early detection of illnesses, for example also by way of self-diagnosis, as well as continuous monitoring of existing illnesses.

The invention accomplishes this task, proceeding from a sensor unit of the type indicated initially, in that an EKG unit for detecting an EKG signal by way of two or more EKG electrodes is provided, whereby at least one EKG electrode of the EKG unit is disposed on the housing surface of the sensor housing, in such a manner that the EKG electrode touches the skin surface in the region of the body tissue covered by the optical measurement unit.

By means of the integration of an optical measurement unit and an EKG unit, according to the invention, a compact sensor unit is created, which yields a plurality of diagnostic measurement values. These can be evaluated individually or in combination, in order to obtain diagnostically conclusive information concerning the health state of the patient being examined, quickly and reliably. The compact sensor unit can be pre-fabricated, as a completely functional part, in large numbers, in cost-advantageous manner, and can be integrated into diagnosis devices of the most varied kinds. The actual measurement can be carried out in particularly simple and convenient manner. For this purpose, the surface of the sensor housing is brought into contact with the skin in the region of the body tissue to be examined, which can take place, for example, by placing a finger of the patient on the housing surface of the sensor unit. The optical measurement and the EKG derivation then take place at the same time, by way of the skin location touching the sensor unit.

According to the invention, the diagnostic sensor unit comprises an optical measurement unit for generating oximetric and/or plethysmographic measurement signals. This makes it possible to monitor the supply of oxygen to the body tissue of the user of the device and/or to generate a volume pulse signal.

The optical measurement unit of the sensor unit according to the invention has a radiation source for irradiating the body tissue being examined with electromagnetic radiation, and at least one radiation sensor for detecting the radiation scattered and/or transmitted by the body tissue. Usual light-emitting diodes or also laser diodes are possible as a radiation source, which emit optical radiation, i.e. light in the corresponding spectral range. It has proven to be particularly advantageous if the radiation absorption in the body tissue being examined is measured, using the device according to the invention, in at least two or, even better, three different light wavelengths, in order to thereby determine the oxygen concentration of the blood and the perfusion of the tissue.

According to a practical embodiment, the optical measurement unit of the sensor unit according to the invention has at least two radiation sensors for detection of the radiation scattered and/or transmitted by the body tissue, whereby the radiation sensors are disposed at different distances from the radiation source. This opens up the possibility of drawing conclusions concerning the distance traveled by the radiation in the body tissue, in each instance. On this basis, the oxygen concentration in the blood and in the tissue in tissue layers that lie at different depths can be investigated. In this connection, advantage can be taken of the fact that the measurement signals from the tissue layers that lie lower down are more strongly influenced by the arterial blood, while the radiation absorption is more strongly influenced by the blood in the capillary vascular system in the regions close to the surface.

An embodiment of the sensor unit according to the invention in which at least two radiation sources are provided, which irradiate different volume regions of the body tissue being examined, is advantageous. In this way, a differential measurement of the light absorption can be implemented in simple manner. This makes it possible to investigate metabolism-induced changes in perfusion of the body tissue being examined, with oxygen-rich or oxygen-poor blood. In this connection, advantage is taken of the fact that the local oxygen consumption changes as a function of the metabolic activity of the tissue. The determination of the changing oxygen consumption in turn permits conclusions with regard to the local energy consumption, which is directly correlated with the oxygen consumption. It is particularly interesting that this in turn permits conclusions concerning the glucose level. Thus, the sensor unit according to the invention advantageously permits non-invasive determination of the blood glucose level, as well.

The two radiation sources of the optical measurement unit of the sensor unit according to the invention should be designed in such a manner that the volume regions irradiated by them, in each instance, are affected differently with regard to the perfusion with oxygen-poor and oxygen-rich blood, respectively. This can be achieved, for example, in that the at least two radiation sources have different spatial emission characteristics. For example, a light emitting diode and a laser that have similar wavelengths (for example 630 nm and 650 nm) can be used as radiation sources. The two radiation sources differ, however, in the aperture angle of their emission. While the light-emitting diode, for example, radiates light into the body tissue being examined at a large aperture angle, the light of the laser diode enters the body tissue at a very small aperture angle. This has the result that different volume regions of the body tissue are detected with the two radiation sources. Because of the large aperture angle, the light-emitting diode detects a larger volume region of the non-perfused epidermis than the laser does. The non-perfused epidermis is practically unaffected by changes in hemoglobin concentration. Accordingly, the intensity of the radiation of the light-emitting diode scattered and/or transmitted by the body tissue is less strongly dependent on a change in the hemoglobin concentration than the intensity of the radiation of the laser. The prerequisite is that the wavelength of the radiation emitted by the two radiation sources, in each instance, is selected in such a manner that the radiation is absorbed to different degrees by the oxyhemoglobin and deoxyhemoglobin, respectively. The wavelength should therefore lie between 600 and 700 nm, preferably between 630 and 650 nm.

According to a practical embodiment of the sensor unit, the at least one radiation source is connected with a light-conducting element, for example an optical fiber. The radiation emitted by the radiation source or the radiation sources, respectively, is conducted to the surface of the sensor housing by way of the light-conducting element. The advantageous possibility exists of coupling the radiation of multiple radiation sources, for example multiple LED chips that are bonded to a common substrate, into a single light-conducting element. In this connection, the different radiation sources can be coupled with the light-conducting element in different ways. In this way, different emission characteristics of the radiation of the different sources into the body tissue to be examined can be achieved.

The sensor unit according to the invention can advantageously be configured to determine a local metabolic parameter from the radiation of the at least two radiation sources scattered and/or transmitted by the body tissue. If oxygen is consumed in the body tissue being examined, oxyhemoglobin is converted to deoxyhemoglobin. By means of a comparison of the radiation of the two radiation sources that comes from the different volume regions of the body tissue, the change in the concentration ratio of oxyhemoglobin and deoxyhemoglobin can be determined. This in turn results in the local oxygen consumption, and finally (indirectly), the blood glucose level.

The EKG unit of the sensor unit according to the invention serves for detecting an EKG signal by way of two or more EKG electrodes. In this way, the functional scope of the sensor unit according to the invention, as compared with conventional systems, is advantageously expanded. The sensor unit according to the invention makes it possible to detect and evaluate pulse-oximetry signals and EKG signals in combination. It is practical if, for this purpose, an evaluation unit for evaluation of the time progression of the optically measured volume pulse signals and the EKG signals is provided. This evaluation unit can be an integral part of the sensor unit. Likewise, it can be provided that the evaluation unit is separate from the sensor unit, whereby the measurement signals are transmitted from the sensor unit to the evaluation unit by way of a suitable data connection. By means of a suitable program control, the evaluation unit is able to automatically recognize the R peaks in the EKG signal. In this way, the precise point in time of the heartbeat is determined automatically. Furthermore, because of a suitable program control, the evaluation unit is able to recognize the maxima in the volume pulse signal. Based on the maxima in the volume pulse signal, the time of arrival of a pulse wave triggered by a heartbeat, at the peripheral measurement location detected by the sensor unit, can be determined. Thus, finally, the time interval between an R peak in the EKG signal and the subsequent maximum in the volume pulse signal can be determined. This time interval is a measure of the so-called pulse wave velocity. On the basis of the pulse wave velocity, a statement about the blood pressure can be made, on the one hand. This is because a shortening in the pulse wave velocity is accompanied by an increase in blood pressure, while a lengthening in the pulse wave velocity permits the conclusion of a reduction in blood pressure. A precise determination of the blood pressure from the pulse wave velocity is not possible, however; only tendencies can be indicated. Furthermore, the pulse wave velocity is dependent on the density of the blood and, in particular, on the elasticity of the blood vessel walls (for example the aorta). In turn, a conclusion concerning arteriosclerosis that might be present can be drawn from the elasticity of the blood vessels. The absolute values of the heart rate, the heart rate variability, and corresponding arrhythmias of the heart can also be included in this evaluation. Thus, arrhythmias such as sinus tachycardia, sinus bradycardia, sinus arrest, and so-called escape beats can be automatically determined. Using the EKG signal, statements concerning the time duration of the atrial contraction of the heart during a heartbeat, the time duration of the heart chamber contraction, as well as the duration of relaxation of the heart chamber, etc., can furthermore be determined. Furthermore, preliminary diagnoses concerning so-called blocks in the line of the electrical excitation signals at the heart (AV block, bundle branch block, etc.) and also with regard to perfusion problems or infarctions are possible. Other irregularities in the pulse progression can be determined using the volume pulse signal. One of the at least two EKG electrodes is disposed on the surface of the sensor housing that also contains the other measurement units, according to the invention. It is practical if the other EKG electrode is disposed in such a manner that the patient can touch the two electrodes with different extremities, for example one of the electrodes with each hand, in each instance.

The invention is based on the recognition, among other things, that the possibility of determining local metabolic parameters is opened up by means of the combination of different diagnosis modalities in a single sensor unit.

For the determination of the local oxygen consumption, for example, the capillary oxygen concentration in the tissue can be determined by means of the sensor unit according to the invention, in addition to arterial oxygen concentration determined by means of oximetry. For this purpose, however, the composition of the body tissue being examined has to be known. Decisive parameters are the local fat content and/or the water content of the body tissue. These parameters can be detected by means of a bioelectrical impedance measurement, for example.

According to a practical embodiment of the invention, a conventional (optical) oximetry unit is therefore combined not just with an EKG unit, but also with a bioelectrical impedance measurement unit, in a single sensor unit. The composition of the body tissue being examined can be determined from the measurement signals obtained by means of the bioelectrical impedance measurement unit. On this basis, the capillary oxygen saturation in the tissue can be determined from the oximetry signals of the sensor unit, for example by means of a suitable program-controlled evaluation unit that is connected with the measurement units of the sensor unit according to the invention. The arterial oxygen saturation (SaO₂) and the venous oxygen saturation (SvO₂) determine the capillary (arteriovenous) oxygen saturation (StO₂) as a function of the tissue being examined. The following holds true:

K*SvO₂+(1−K)*SaO₂=StO₂

where K is a tissue-dependent correction factor that depends on the volume ratio of arteries to veins in the tissue being examined. On average, this value lies slightly below 0.5. The value decisive for the tissue, in each instance, can be determined, according to the invention, by means of a bioelectrical impedance measurement, in order to then determine the venous oxygen saturation from the above formula. The sensor unit according to the invention can be used to determine the perfusion V, i.e. the perfusion-related volume variation of the body tissue being examined. According to the equation

VO₂=V*(SaO₂−SvO₂)

the local oxygen consumption VO₂ can finally be calculated, which represents a measure of the metabolic activity at the measurement location.

It is practical if feed or measurement electrodes are disposed on the housing surface of the sensor housing for the bioelectrical impedance measurement, so that the bioimpedance measurement can take place at the same time with the oximetry and EKG measurement. In this connection, the same region of the body tissue, namely the location where the patient is touching the surface of the sensor housing, is covered by all the measurement modalities at the same time.

According to another advantageous embodiment, the sensor unit according to the invention comprises an integrated temperature or heat sensor. This sensor can be used to determine the local heat production. In the simplest case, the temperature sensor (for example an NTC element) is configured to measure the surface temperature of the skin at the measurement location. Preferably, a heat measurement that is location-resolved, time-resolved, and depth-resolved is possible at the measurement location. Based on the heat exchange, a conclusion can be drawn with regard to the local metabolic activity. Furthermore, the heat sensor is suitable for determining the local perfusion. With regard to more detailed background information concerning heat measurement, reference is made to the publication by Nitzan et al. (Meir Nitzan, Boris Khanokh, “Infrared Radiometry of Thermally Insulated Skin for the Assessment of Skin Blood Flow,” Optical Engineering 33, 1994, No. 9, p. 2953 to 2956). In total, the heat sensor provides data that can advantageously be used to determine metabolic parameters.

The combination of the aforementioned measurement methods, namely oximetry, EKG measurement, temperature or heat measurement, and—optionally—bioelectrical impedance measurement, according to the invention, is particularly advantageous. All the measurement signals can be evaluated and combined by means of the program-controlled evaluation unit mentioned above, using a suitable algorithm. By means of the combination of the different measurement modalities, great effectiveness and reliability in the recognition of pathological changes are achieved. It is advantageous that all the parameters can be combined to yield a global index that can easily be interpreted by the user and gives him direct and well-founded information concerning his general state of health.

The combination of the different measurement modalities that can be combined in the sensor unit according to the invention, as described above, is furthermore advantageous because this makes non-invasive indirect measurement of the glucose concentration possible. A possible method of procedure in the determination of the blood glucose level by means of the device according to the invention will be explained in greater detail below:

The sensor unit according to the invention serves to measure data that are influenced by the metabolism. It is directly evident that in this connection, the energy metabolism and the composition of the nutrients taken in by a patient being examined play a large role. The nutrients that are involved in the metabolism are known to be essentially carbohydrates, fats, and proteins. For further processing, carbohydrates are converted to glucose, proteins are converted to amino acids, and fats are converted to fatty acids. The energy carriers in turn are converted in the cells of the body tissue, together with oxygen, to produce ATP (adenosine triphosphoric acid), giving off energy. ATP is the actual energy carrier of the body itself. The use of glucose to produce ATP is preferred. However, if the production of ATP from glucose is inhibited (for example due to a deficiency of insulin), increased fatty acid oxidation takes place, instead. However, the oxygen consumption is different in this process.

The reaction of the metabolism of the human body to an intake of nutrients depends, as was mentioned above, on the composition of the nutrients, in characteristic manner. For example, the vascular system of the body reacts as a function of how much energy the body requires to digest the foods that are consumed. The reaction of the body to nutrient intake can be determined on the basis of the pulse wave velocity, which can be determined using the sensor unit according to the invention, as well as on the basis of the blood pressure amplitude and the pulse. The pulse wave velocity, as well as the blood pressure amplitude and the pulse, change as soon as the intake of nutrients begins. The maxima and the points in time of the maxima, in each instance, are influenced, in this connection, by the nutrient composition. The progression and the absolute height of the pulse wave velocity, blood pressure amplitude, and pulse can be used to determine the composition of the nutrients taken in. The metabolism of the human body is determined essentially by the glucose metabolism in the normal state, i.e. at rest and in the so-called thermoneutral zone. For this reason, the glucose concentration in the cells of the body tissue in this normal state can be described as a pure function of heat production and oxygen consumption. The following applies:

[Glu]=f₁(ΔT, VO₂),

where [Glu] stands for the glucose concentration. The heat production ΔT can be determined by means of the heat sensor of the sensor unit according to the invention, for example from the difference between the arterial temperature and the temperature that the skin surface would reach in the case of perfect thermal insulation (ΔT=T_{∞}−T_{artery}). f₁ (ΔT, VO₂) indicates the functional dependence of the glucose concentration on the heat production and on the oxygen consumption. The oxygen consumption results from the difference between venous and arterial oxygen saturation and perfusion, as was explained above. To determine the glucose concentration during or immediately after nutrient intake, however, a correction term has to be taken into consideration, which reproduces the proportion of the fat metabolism in the energy metabolism. The following then applies:

[Glu]=f₁(ΔT, VO₂)+X*f₂(ΔT, VO₂).

X is a factor that is negative after nutrient intake. In this connection, X depends on the composition of the nutrients taken in. In particular, X depends on the ratio at which fat and carbohydrates are involved in the metabolism. The factor X can be determined, as was described above, using the time progression of the pulse wave velocity. X is 0 if pure carbohydrates or glucose are consumed directly. The amount of X increases, the greater the proportion of fat in the nutrients taken in. To determine the correction factor X from the time progression of the pulse wave velocity, the blood pressure amplitude and/or the pulse, a calibration for adaptation to the user of the device, in each instance, will normally be necessary. f₂ (ΔT, VO₂) indicates the functional dependence of the glucose concentration on the heat production and on the oxygen consumption, for the fat metabolism.

The sensor unit according to the invention (in combination with the integrated or separate evaluation unit mentioned above) can thus be used to determine the local glucose concentration from the local oxygen consumption and the local heat production. For this purpose, the sensor unit must have the suitable measurement modalities. The determination of oxygen consumption, as was explained above, can take place by means of a combination of oximetry with a bioelectrical impedance measurement. To determine the heat production, the aforementioned heat sensor is then additionally required. Finally, in order to be able to calculate the glucose concentration according to the functional relationship indicated above, the correction factor X should also be determined, for example from the time progression of the pulse wave velocity. This can take place, as was also explained above, by means of a combined measurement of EKG signals and plethysmographic signals. Therefore, in order to determine the glucose concentration, it is practical if the sensor unit according to the invention combines a pulse oximeter, an EKG unit, a bioelectrical impedance measurement unit, as well as a heat sensor.

The method outlined above at first only allows a determination of the intracellular glucose concentration. The following relationship with the blood glucose concentration exists, in simplified form:

[Glu]_{cell}=a+b*ln(c*[Glu]_{blood})

The constants a, b, and c depend on the individual physiology of the patient being examined. Thus, the evaluation unit connected with the sensor unit can furthermore be set up to determine the blood glucose level from the local glucose concentration, whereby parameters that depend on the physiology of the patient have to be taken into consideration. These parameters can be determined by means of corresponding calibration, for example by means of a comparison with blood glucose values determined invasively, in conventional manner.

For practical use, the sensor unit according to the invention can be connected with any desired program-controlled device, for example a computer, a mobile telephone, a handheld, etc., whereby the functions for evaluation of the detected measurement signals are implemented by means of software that runs on the program-controlled device. Because of the small size of the sensor unit, this unit can also be integrated into any desired accessory, for example eyeglasses, a wristwatch, a piece of jewelry, or the like, or into an article of clothing (so-called “smart clothes”). In the case of this embodiment, the data processing electronics that are present in the program-controlled device, in any case, for example, are used to process the measurement signals obtained by means of the sensor unit. This can easily be done by means of making the corresponding software available. At the same time, the diagnostic data determined by means of the software can be stored in memory. This makes it possible to follow up and document the progression of an illness and the effects of corresponding therapy. It is practical that remote data transmission of the diagnostic data detected and evaluated by means of the sensor unit can also take place. Data transmission can take place, for example, by way of a data network (for example the Internet). Alternatively, the diagnostic data can be transmitted by way of a mobile radio network, if the sensor unit according to the invention is integrated into a mobile telephone, for example. The raw measurement signals or the evaluated diagnostic data can be transmitted, for example, to a central location (“healthcare center”) for a more detailed analysis and documentation, and for monitoring of the development over time of individual values. There, the data are evaluated, for example, by means of suitable analysis algorithms, if necessary taking into consideration patient data stored there (including information concerning chronic illnesses or prior illnesses). The result, in turn, can be sent back to the mobile telephone, for example, by way of the data network or communication network, in each instance, in order to inform the user of the device accordingly, about his state of health. From the central location, other targeted measurements by means of the sensor unit according to the invention can also be initiated, if necessary. Furthermore, for the purpose of an expanded anamnesis, queries to the patient, based on the evaluation results, can be transmitted by way of the data network or communication network. The data and evaluation results can automatically be transmitted to a treating physician. If indications of a medical emergency become evident from the measurement and evaluation results, the required measures (for example automatic alarm to emergency services) can be initiated immediately. Another advantage of remote data transmission is that the required software for evaluation of the measurement signals does not have to be implemented in the device itself, but rather merely has to be kept on hand and administered at the central location where the data are received.

In the case of pulse oximetry measurements, the contact pressure of the body tissue (for example the finger) on the optical sensor has a significant influence on the measurement signals. Accordingly, it can be practical to equip the sensor unit according to the invention with means for determining the contact pressure of the body tissue. This can be a conventional pressure sensor, for example in the form of a piezo-resistive element. Optical methods for determining the contact pressure are also possible. Likewise, it is possible to determine the contact pressure from the (pulse oximetry) signals themselves, since the contact pressure has a characteristic effect on the measurement signals. The contact pressure that is determined can then be taken into consideration in the further evaluation of the measurement signals, in order to compensate the influence of the contact pressure on the perfusion, for example.

According to the invention, the optical measurement unit, the EKG unit, and, if applicable, the temperature or heat sensor are accommodated in a common sensor housing. It is practical if a planar EKG electrode, for example in the form of an electrically conductive foil or an electrically conductive sheet is configured on the top of the sensor housing, which has at least one recess for passage of the radiation of the radiation emitted by the at least one radiation source. It is practical if the planar EKG electrode has another recess for the temperature or heat sensor. The radiation source, the radiation sensor, and the temperature or heat sensor can be disposed on a common board within the sensor housing. Thus, the required measurement modalities are combined in the sensor housing, which forms a unit that can be easily and flexibly integrated into any desired diagnosis device. The sensor housing can have dimensions of less than 1 cm×1 cm×1 cm, in order to be able to be used easily and flexibly in the sense of the invention. On the top of the sensor housing, furthermore, at least one additional planar electrode can be formed, which serves as a feed or measurement electrode of the impedance measurement unit, in order to additionally allow a bioelectrical impedance measurement. In this connection, it is practical to use the EKG electrodes, which are present in any case, also as feed or measurement electrodes for the bioimpedance measurement. In total, an extremely compact, integrated sensor unit is obtained, which contains different measurement modalities. The same region of the body tissue to be examined (for example a fingertip of a patient that touches the surface of the sensor housing) can be covered by all the measurement modalities, in order, as was explained above, to examine the metabolism and the cardiovascular system of the patient at the same time. This permits carrying out a measurement in particularly simple and effective manner.

Exemplary embodiments of the invention will be explained in greater detail below, making reference to the drawings. These show:

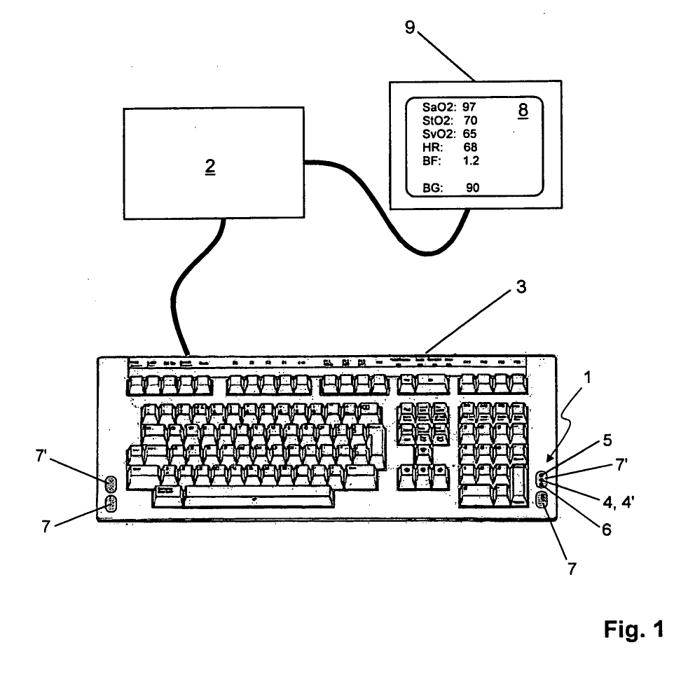

FIG. 1 schematic view of the integration of the sensor unit according to the invention into a computer keyboard;

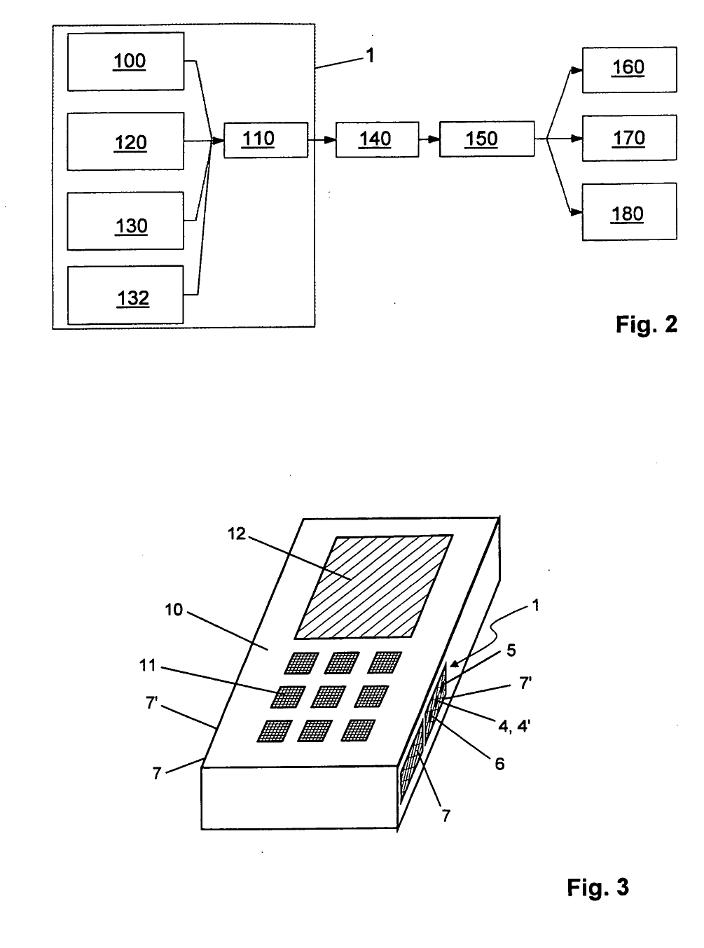

FIG. 2 representation of the function of the sensor unit according to the invention, using a block diagram;

FIG. 3 schematic view of the integration of the sensor unit into a mobile telephone;

FIG. 4 illustration of the diagnostic sensor unit;

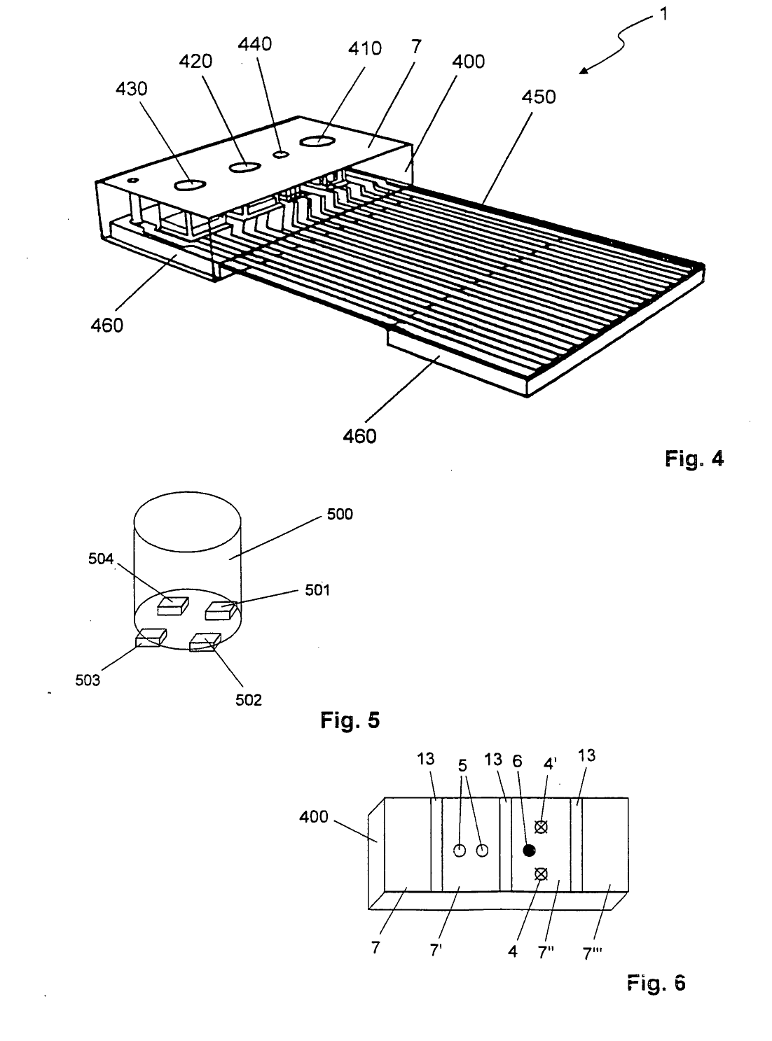

FIG. 5 light-conducting element of the sensor unit according to the invention;

FIG. 6 top view of another exemplary embodiment of the sensor unit according to the invention.

FIG. 1 shows a sensor unit according to the invention referred to as a whole with the reference number 1, which is integrated into a computer system consisting of a computer 2 and a keyboard 3. The sensor unit 1 has different measurement modalities, which are accessible at the user interface of the keyboard 3. The user of the computer system touches it with his fingertips in order to perform a measurement. Light sources 4, 4′, for example in the form of light-emitting diodes, are integrated into the sensor unit 1, and are able to emit light at different wavelengths. For this purpose, different light-emitting semiconductor elements are accommodated in a common sensor housing (not shown in FIG. 1). It is also possible to use light-wave conductors, in order to guide the light from different light sources to the user interface of the keyboard 3. Furthermore, the sensor unit 1 comprises one or more photosensors 5. The photosensors are disposed in the immediate vicinity of the light source 4 or 4′, respectively. The sensors 5 receive the light from the light source 4 or 4′ scattered in the tissue on the fingertip of the user. Furthermore, a heat sensor 6 is provided directly next to the light source 4 or 4′. In this way, it is guaranteed that the determination of the perfusion based on the heat measurement takes place at the same measurement location as the optical measurement. Furthermore, a total of four electrodes 7 or 7′, respectively, for measuring the bioelectrical impedance, are provided on the surface of the sensor unit 1. The user of the device touches two electrodes 7 and 7′, respectively, at the same time, with a hand. One of the two contact surfaces serves to apply an electrical current at the measurement location, while the other contact surface is used for a voltage measurement. In this way, it is ensured that the measurement results are not influenced by the contact resistances of the measurement electrodes. The two electrodes indicated with the reference number 7 are furthermore used as EKG electrodes of an EKG unit that is also integrated into the sensor unit 1. The two electrodes are touched with the fingertips, in each instance, so that a two-point derivation (arm to arm measurement) is obtained. The measurement signals recorded by means of the sensor unit 1 integrated into the keyboard 3 are processed by means of the computer 2. The physiological parameters obtained in this manner are then output on a display surface 8 of a monitor 9 connected with the computer 2. The arterial (SaO₂), capillary (StO₂), and venous (SvO₂) oxygen saturation are displayed. Furthermore, the heart rate determined (HR) and the fat content of the tissue (BF) are displayed. Finally, a blood glucose value (BG) is also displayed. The user can determine the physiological parameters that are of interest to him at any time. For this purpose, he merely places the fingers with which he normally operates the keyboard 3 onto the electrodes 7, 7′. The parameters are then displayed immediately after processing of the measurement signals by means of the computer 2, using the monitor 9. The user of the device 1 therefore practically does not have to interrupt his work on the computer 2 in order to determine the physiological parameters.

In the exemplary embodiment of the sensor unit 1 shown in FIG. 1, two radiation sources 4 and 4′ are provided, which irradiate different volume regions of the body tissue being examined. For this purpose, the two radiation sources 4 and 4′ have different spatial emission characteristics, namely different emission angles. The radiation source 4 is a light-emitting diode, while the radiation source 4′ is a laser, for example a so-called VCSEL laser (English: “vertical cavity surface emitting laser”). Both the light-emitting diode 4 and the laser 4′ emit light having a very similar wavelength (for example 630 nm and 650 nm), but with different aperture angles (for example 25° and) 55°). With the array shown in FIG. 1—as explained above—a differential measurement of metabolism-induced changes in the oxygen content in the blood is possible. For this purpose, the wavelength of the radiation emitted by the two radiation sources 4 and 4′, in each instance, must lie in a range in which the light is absorbed to different degrees by oxyhemoglobin and deoxyhemoglobin. For an absolute measurement of the oxygen content of the blood (oxygen saturation), other radiation sources (not shown in FIG. 1) must be present, whose wavelength lies in a spectral range in which the light absorption of oxyhemoglobin and deoxyhemoglobin is essentially the same (so-called isosbectic point). The light emitted by the light-emitting diode and the laser, respectively, can be guided to the corresponding location on the user interface of the keyboard by means of corresponding light-guide fibers. In this case, the corresponding fiber ends are shown in FIG. 1 with the reference symbols 4 and 4′. It is possible to couple the light-emitting diode and the laser to the corresponding fibers in such a manner that they emit light into the body tissue to be examined at the desired different aperture angle. Accordingly, different volumes of the body tissue are examined with the two radiation sources. Because of the greater aperture angle, the proportion of the non-perfused epidermis in the body tissue examined by means of the light-emitting diode is greater than in the case of the laser. The light scattered and partly absorbed in the body tissue, both of the radiation source 4 and of the radiation source 4′, is detected by means of the sensors 5. The sensors 5 do not have to be disposed directly on the surface of the sensor unit 1. Instead, the light can be passed to the sensors disposed in the interior of the sensor unit 1 by means of light-guide fibers. For a differentiation of the light of the radiation source 4 from the light of the radiation source 4′, the two light sources 4 and 4′ can be operated with different time modulation, whereby the signals detected by means of the sensors 5 are demodulated accordingly. Alternatively, it is possible to differentiate the radiation of the two radiation sources 4 and 4′ on the basis of the different wavelength. The radiation intensity of the radiation emitted by the radiation sources 4 and 4′ is weakened with the path length when passing through the body tissue, whereby the relationship of the intensity weakening with the concentration of the absorbed substance (oxygenated hemoglobin) is given by the known Lambert-Beer law. By means of the sensors 5 shown in FIG. 1, the parameters of the intensity weakening that are of interest can be determined, specifically separately for the volume regions of the body tissue covered by the radiation sources 4 and 4′, in each instance. The parameters of the intensity weakening that are to be assigned to the different radiation sources 4 and 4′ can be put into relation with one another by means of a suitably program-controlled evaluation unit, in order to carry out a differentiated measurement in this way. In the simplest case, quotients are calculated, in each instance, from the parameters of the intensity weakening of the radiation of the two radiation sources 4 and 4′. From changes in these quotients, it is then possible to draw conclusions concerning changes in the metabolism. If, for example, the blood glucose level increases after an intake of nutrients, correspondingly more glucose gets into the cells of the body tissue (after a certain time delay) and is converted there. In this connection, oxygen is used up. The cells get this oxygen by way of the blood. In this connection, the oxygenated hemoglobin becomes deoxygenated hemoglobin, by giving off oxygen. Accordingly, the ratio of deoxygenated hemoglobin to oxygenated hemoglobin increases. Because of the different aperture angles of the radiation of the radiation sources 4 and 4′, the changes in hemoglobin concentration have different effects on the intensity weakening, in each instance. Thus, changes in the hemoglobin concentration can be detected from the quotient of the parameters of the intensity weakening. This makes it possible to draw a conclusion concerning oxygen consumption indirectly. Since the oxygen consumption in turn depends on the blood glucose level, the blood glucose level can also be determined by means of the differential measurement of the radiation absorption that has been explained. As a practical supplement, parallel to the optical measurement, a bioimpedance analysis is carried out, for which purpose the electrodes 7 and 7′ shown in FIG. 1 are provided. The purpose of the bioimpedance measurement is primarily the determination of the local perfusion. This can be used as an additional parameter in the determination of the oxygen consumption and thus also of the blood glucose level. Different aperture angles of the radiation can also be generated with only one radiation source 4, by means of using corresponding optical elements (for example beam splitters, lenses, etc.).

FIG. 2 schematically shows the structure of the sensor unit 1 according to the invention as a block diagram. The sensor unit 1 comprises an optical measurement unit 100 for optical measurement of the oxygen concentration in the vascular system of the body tissue at the measurement location, in each instance. The oximetry and plethysmography signals recorded by means of the optical measurement unit 100 are passed to an analysis unit 110. Another essential component of the device 1 is a heat measurement unit 120 for determining the local heat production. The heat measurement unit 120 is a special heat sensor that insulates the body location being examined, in each instance. This location can therefore only absorb or give off heat by means of the blood stream. For this reason, it is possible to determine the perfusion and the heat production by means of the time-resolved measurement of temperature. In the case of strong perfusion, the body location being examined reaches its maximal temperature in a very short time. In the case of little perfusion, this takes longer. In addition, by way of extrapolation of the measured temperature, it is possible to draw conclusions concerning the arterial temperature, since the temperature at the measurement location is determined only by the arterial temperature and by the local heat production. The measurement signals recorded by the heat measurement unit 120 are also passed to the analysis unit 110 for further processing. Furthermore, the sensor unit comprises an impedance measurement unit 130, which serves to detect local tissue parameters by means of a bioelectrical impedance measurement. The measurement signals of the impedance measurement unit 130 are also processed by means of the analysis unit 110. Finally, according to the invention, an EKG unit 132 for detecting an EKG signal is also provided. The EKG unit 132 is also connected with the analysis unit 110, for processing of the EKG signals. The optical measurement unit 100 has the light sources 4, as well as the light sensors 5 of the sensor unit 1 shown in FIG. 1 assigned to it. The heat measurement unit 120 is connected with the heat sensor 6. The impedance measurement unit 130 detects measurement signals by way of the electrodes 7 and 7′, respectively, of the sensor unit 1. The analysis unit 110 carries out pre-processing of all the measurement signals. For this purpose, the signals pass through a band-pass filter, in order to filter out interference in the range of the network frequency of 50 or 60 Hz, respectively. Furthermore, the signals are subjected to noise suppression. After passing through the analysis unit 110, the processed signals of the optical measurement unit 100, the heat measurement unit 120, the impedance measurement unit 130, and the EKG unit 132 reach an evaluation unit 140. The evaluation unit 140 is responsible for calculating the parameters essential for the diagnosis from the measurement signals. The functions of the evaluation unit 140 are essentially implemented by means of software. Therefore the evaluation unit 140 is not an integral part of the actual sensor unit 1 in the exemplary embodiment shown. First, the composition of the body tissue being examined (water content, fat content, etc.) is calculated from the time-dependently recorded measurement signals of the impedance measurement unit 130. The arterial oxygen saturation and—based on the tissue parameters determined previously, on the basis of the impedance measurement—the capillary oxygen saturation are calculated from the signals of the optical measurement unit 100. Furthermore, the perfusion and the arterial temperature are determined from the measurement signals of the heat measurement unit 120 and from the plethysmographic data that can be derived from the time-dependent impedance measurement. The pulse wave velocity is determined from the signals of the EKG unit 132 and those of the optical measurement unit 100. Finally, the venous oxygen saturation, and from it other metabolic parameters, particularly the local oxygen consumption and the glucose concentration at the measurement location, are calculated by means of the evaluation unit 140, from the results of all the calculations carried out previously. The calculation results are interpreted by means of a diagnosis unit 150. The diagnosis unit 150, which is also implemented as software on the computer 2, serves for evaluating the local metabolic parameters calculated by means of the evaluation unit 140. The evaluation unit 140 and the diagnosis unit 150 are connected with a graphics unit 160, which in turn controls the monitor 9, to display the measurement results. The data obtained can be stored in a memory unit 170, specifically while simultaneously storing the date and the time of day of the measurement, in each instance. Furthermore, an interface unit 180 is provided, which serves to connect the computer 2 with a data network for transmission of the calculated physiological parameters. By way of the interface unit 180, all the data and parameters, particularly also the data and parameters stored in the memory unit 170, can be transmitted to a PC of a treating physician, which is not shown in any detail. There, the data can be analyzed in greater detail. In particular, data and parameters recorded with the sensor unit 1 over an extended period of time can be investigated with regard to changes, in order to be able to draw conclusions concerning the development of an existing illness from this.

FIG. 3 shows a second example of use for the sensor unit 1 according to the invention, namely in a mobile telephone 10. On the front of the device 10, the usual operating keys 11 can be seen. The diagnostic measurement sensors of the sensor unit 1 are integrated, flush, into the side surfaces of the housing of the device 10. The user of the mobile telephone 10 touches them with his fingers in order to perform a measurement. A total of four electrodes 7 or 7′ for measuring the bioelectrical impedance is provided on the lateral housing surfaces of the mobile telephone 10. The user of the mobile telephone 10 touches two electrodes 7 or 7′, respectively, at the same time, with a hand. The two electrodes are touched with the fingertips, in each instance, so that a two-point derivation (arm to arm measurement) is obtained. The measurement signals recorded by means of the different sensors integrated into the sensor unit 1 of the mobile telephone 10 are processed by means of the microprocessor (not shown in any detail) of the mobile telephone 10. The physiological parameters obtained in this manner are then output on a display 12 of the mobile telephone 10. The user can determine the physiological parameters that are of interest to him at any time. For this purpose, he merely places the fingers with which he normally activates the keys 11 on the electrodes 7, 7′. The software controller of the mobile telephone 10 automatically recognizes the touch and starts the measurement. The parameters are then displayed immediately after processing of the measurement signals by means of the microprocessor of the mobile telephone 10, by means of the display 12. The function of the mobile telephone 10, which is configured as a medical device by means of integration of the sensor unit 1, is essentially based on the indirect method for non-invasive determination of the blood glucose value as described above, in which the effect of the glucose, i.e. the energy conversion of the physiological reactions in the body initiated by the glucose is examined. Reference is made to the corresponding description for an explanation of the exemplary embodiment shown in FIG. 1. Similar to the keyboard 3, the light source 4, 4′ and the sensors 5 do not have to be disposed directly on the housing surface in the case of the mobile telephone 10, either. Instead, the light can be guided from or to the housing surface by way of light-guide fibers, whereby the actual light sources and sensors, respectively, are situated in the interior of the housing. Multiple light sources and/or sensors can be coupled to a single light-guide fiber.

FIG. 4 illustrates the design of the diagnostic sensor unit 1 according to the invention. The different measurement units of the sensor unit 1 are integrated into a sensor housing 400 that has very small outside dimensions. A planar EKG electrode 7 is disposed on the surface of the housing 400, composed of a thin, electrically conductive foil. When the sensor unit is built into a computer keyboard or mobile device, the sensor housing 400 is disposed in such a manner that the user can touch the EKG electrode 7 and another electrode (not shown in FIG. 4) for EKG derivation with different extremities. It is practical if the EKG electrode is a thin stainless steel foil. The small construction size of the micro-housing, at 5 mm (W)×8 mm (L)×1.8 mm (H) in the exemplary embodiment shown, allows flexible and cost-advantageous installation of the sensor unit into different housings of different devices available on the market. For simultaneous determination of the oxygen saturation in arterial blood, an optical measurement unit, namely a pulse oximeter, is integrated into the sensor housing 400. This comprises two or more optical radiation sources, whose radiation can pass through a recess 410 in the EKG electrode 7. Furthermore, the pulse oximeter comprises two optical radiation sensors, for example in the form of photodiodes. The light scattered in the body tissue (for example of a finger laid onto the electrode 7) falls onto the radiation sensors through two recesses 420 and 430 in the electrode 7. The recesses 420 and 430 are disposed at different distances from the recess 410. In the sensor unit, the light from two or more optical radiation sources (for example light-emitting diodes) within the housing 400 is coupled into a light-guide fiber or into a suitable light-conducting body, so that only one recess 410 for all the radiation sources is situated on the top of the micro-housing, and the light of all the radiation sources of the sensor unit is passed into the body tissue to be examined at the same location. The photodiodes are individually coupled to a light-guide fiber or to a suitably configured light-conducting body, in each instance. The optical measurement unit allows simultaneous measurement of the oxygen saturation of the blood circulating in the body tissue being examined, and of the volume pulse. It is practical if not only light-emitting diodes, but also other radiation sources, such as, for example, vertical cavity surface emitting lasers (VCSEL) are used for this purpose. For simultaneous determination of the thermal properties of the tissue being examined, a temperature sensor, namely a thermistor, is integrated into the sensor housing. For this sensor, another recess 440 in the EKG electrode 7 is provided. The thermistor is disposed in the sensor housing 400 in such a manner that it has good thermal contact with the body tissue being examined. In the exemplary embodiment shown, the thermistor is situated between the recess 410 for the light-guide fiber of the optical radiation sources and the recess 420 for the light-guide fibers of the first photodiode. The sensor unit can easily be supplemented with an impedance measurement unit. For this purpose, at least one additional planar electrode (not shown in FIG. 4) has to be configured on the top of the sensor housing 400, which then serves as a feed or measurement electrode of the impedance measurement unit. It is practical if the same measurement electrodes are used to detect the bioimpedance signal and the EKG signal. For the electrical contact of the sensor unit (for example with the electronics of a mobile telephone), the sensor housing 400, with all the integrated measurement units, is mounted directly onto a ribbon cable 450 with a suitable conductor track so that simple electrical assembly of the sensor unit 1, using the ribbon cable 450, is possible. The ribbon cable 450 can have reinforcements 460 at suitable locations, for stabilization purposes.

FIG. 5 shows the light-conducting element 500 mentioned above with regard to FIG. 4 with a total of four LED chips 501, 502, 503, and 504 coupled onto the underside of the element 500, which form light sources of the optical measurement unit of the sensor unit 1 according to the invention. By means of the one single light-conducting element 500, the emitted radiation of all the LEDs 501, 502, 503, and 504 is passed to the surface of the sensor housing 400. The four LEDs 501, 502, 503, and 504 are bonded onto a substrate (not shown), for example a PCB, next to one another.

FIG. 6 shows another exemplary embodiment of the invention, whereby a total of four electrodes 7, 7′, 7″, and 7′″ are disposed on the top of the sensor housing 400, which can be used as feed and measurement electrodes for (local) bioelectrical impedance measurement as well as for EKG derivation. The electrodes 7, 7′, 7″, and 7′″ are separated from one another by means of insulating strips 13.

1. Diagnostic sensor unit for non-invasive detection of at least one physiological parameter of body tissue close to the skin surface, having an optical measurement unit (100) that comprises at least one radiation source (4) for irradiating the body tissue to be examined, and at least one radiation sensor (5) for detecting the radiation scattered and/or transmitted by the body tissue, whereby the at least one radiation source (4) and the at least one radiation sensor (5) are disposed in a common sensor housing (400), wherein
an EKG unit (132) for detecting an EKG signal by way of two or more EKG electrodes (7) is provided, whereby at least one EKG electrode (7) of the EKG unit (132) is disposed on the housing surface of the sensor housing (400), in such a manner that the EKG electrode (7) touches the skin surface in the region of the body tissue covered by the optical measurement unit (100).

2. Diagnostic sensor unit according to claim 1, wherein a temperature or heat sensor (6) is disposed in or on the sensor housing (400).

3. Diagnostic sensor unit according to claim 1, wherein the at least one EKG electrode (7) is configured as a planar foil or sheet of electrically conductive material, whereby the EKG electrode (7) has at least one recess (410) for passage of the radiation emitted by the at least one radiation source (4) into the body tissue to be examined.

4. Diagnostic sensor unit according to claim 2, comprising at least one other recess (440) for the temperature and heat sensor (6).

5. Diagnostic sensor unit according to claim 1, further comprising a bioelectrical impedance measurement unit (130), whereby at least one feed or measurement electrode of the impedance measurement unit (130) is disposed on the housing surface of the sensor housing.

6. Diagnostic sensor unit according to claim 5, wherein at least one of the EKG electrodes (7) is a feed or measurement electrode of the bioelectrical measurement unit (130), at the same time.

7. Diagnostic sensor unit according to claim 1, wherein the sensor housing (400) has dimensions of less than 1 cm×1 cm×1 cm.

8. Diagnostic sensor unit according to claim 1, wherein the optical measurement unit (100) has at least two radiation sensors (5) for detection of the radiation scattered and/or transmitted by the body tissue, whereby the radiation sensors (5) are disposed at different distances from the radiation source (4).

9. Diagnostic sensor unit according to claim 1, wherein at least two radiation sources (4, 4′) are provided, which irradiate different volume regions of the body tissue being examined.

10. Diagnostic sensor unit according to claim 9, wherein the at least two radiation sources (4, 4′) have different spatial emission characteristics.

11. Diagnostic sensor unit according to claim 1, wherein the at least one radiation source (4) is connected with a light-conducting element (500) that guides the radiation emitted by the radiation source (4) to the surface of the sensor housing (400).

12. Diagnostic sensor unit according to claim 11, wherein at least two radiation sources (4, 4′) are connected with the light-conducting element (500) that guides the radiation of the at least two radiation sources (4, 4′) to the surface of the sensor housing (400).

13. Diagnostic sensor unit according to claim 1, further comprising an electrical plug-in connection by way of which the sensor unit can be connected with a device (10) of entertainment or communications technology, or with some other portable device or accessory.

14. Diagnostic sensor unit according to claim 13, wherein the device (10) is a mobile device, particularly a notebook, a laptop, a palmtop, or a handheld.

15. Diagnostic sensor unit according to claim 1, further comprising a means for determining the contact pressure of the body tissue on the surface of the sensor housing (400).
